(19) United States
(12) Patent Application Publication
(10) Pub. No.: US 2004/0030097 A1
(43) Pub. Date: Feb. 12, 2004

(54) Peptide mimetics

(76) Inventors: Luis Serrano, Heidelberg (DE); Helena Maria Domingues, Munchen (DE)

Correspondence Address: WENDEROTH, LIND & PONACK, L.L.P., 2033 K STREET N. W., SUITE 800, WASHINGTON, DC, 20006-1021, US

(21) Appl. No.: 10/344,260

(22) PCT Filed: Aug. 9, 2001
(86) PCT No.: PCT/IB01/01705

(30) Foreign Application Priority Data
Aug. 9, 2000 (GB) 0019638.6

(51) Int. Cl. C07K 14/00

(52) U.S. Cl. 530/350

(57) ABSTRACT

The invention relates to peptide mimetics of cytokine molecules that comprise an atypical helix-turn-helix motif that has been mutated to incorporate one or more amino acid residues from the active site of said cytokine molecule. In particular, the invention relates to peptide mimetics of type I cytokine molecules such as interleukin 4, (IL-4), human growth hormone (HGH) and interleukin 2 (IL-2).

The invention relates to peptide mimetics of cytokine molecules. In particular, the invention relates to peptide mimetics of type I cytokine molecules such as interleukin 4, (IL-4), human growth hormone (HGH) and interleukin 2 (IL-2).

Cytokines are small proteins of between around 8 and 80 kDa that have a central role in both positive and negative regulation of immune reactions, as well as in integrating these reactions with other physiological compartments such as the endocrine and hemopoietic systems.

Well over one hundred different human cytokines have now been identified, that possess a wide variety of different functions. These molecules act by binding to specific receptors at the cell membrane, so initiating a signalling cascade that leads to the induction, enhancement or inhibition of a number of cytokine-regulated genes. There are various different types of cytokines, including the interleukins, interferons, colony stimulating factors, tumour necrosis factors, growth factors and chemokines. These cytokines function together in a complex network in which the production of one cytokine generally influences the production of, or response to, several other cytokines.

Clinically, cytokines have important roles in several areas of medicine, including their use as anti-inflammatories, and as agents used to treat a number of cancers, including non-Hodgkin's lymphoma, multiple myeloma, melanoma and ovarian cancer. Cytokines also have applications in the treatment of HIV, multiple sclerosis, asthma and allergic diseases.

The activity of cytokines can be inhibited by preventing the interaction of the specific cytokine with its receptor system, thereby suppressing the intracellular signals that are responsible for the cytokine's biological effects. The strategies that are available to block cytokine-receptor interactions generally involve the use of monoclonal antibodies against the cytokine or against its receptor. In addition, soluble receptors and cytokine receptor antagonists may be used (see Finkelman et al 1993; Rose-John and Heinrich, 1994). Receptor antagonists are mutants of the wild type cytokine that are able to bind to cytokine receptors with high affinity, but which are not able to induce signal transduction and therefore do not generate a biological response. In the case of IL-4, two efficient antagonists have been reported in the literature that bind to the IL-4 receptor alpha with a Kd similar to that of the wild type protein, but which are unable to recruit a second receptor component.

However, the therapeutic potential of soluble receptors and monoclonal antibodies has been shown to be rather limited, due to the high doses that are required, and the possible immunogenicity of these proteins (Finkelman et al 1993; Maliszewski et al 1994).

For these reasons, a great deal of attention has been devoted to the possible utilisation of cytokine-derived antagonists as therapeutic molecules. This new generation of bio-pharmaceuticals is expected to be of lower toxicity as compared to other substances (Buckel, 1996).

However, the therapeutic potential of conventional cytokine-derived antagonists is diminished by virtue of the fact that these proteins tend not to induce the desired biological response efficiently and must therefore be administered in large quantities. Furthermore, most are difficult to produce in large amounts in a cost-effective way, because they tend to form inclusion bodies when overexpressed in E. coli, and they refold in vitro in very low yields.

A number of peptide mimetics of cytokines have been described previously. For example, the rational design of peptide mimetics of IL-4 has been reported. These molecules bind IL-4Rα with K_{d}s ranging between 100 mM and 5 μM (Domingues et al., 1999). These peptides were designed by inserting the IL-4 epitope for IL-4Rα into the helices of the parallel coiled-coil domain of the yeast transcription factor GCN4 (O'Shea et al., 1991). In this way, short peptides have been obtained, 31 residues long, that dimerize in solution and form a dimeric coiled-coil structure, bearing the IL-4 functional residues in a spatial orientation suitable for interaction with IL-4Rα. Such rational design strategies have allowed the design of antagonists with reasonable affinities (μM range).

Phage display technology has also been used in the creation and screening of vast peptide libraries (Cwirla et al., 1990), whereby peptides displayed on the surface of phage particles are screened for binding to a particular target. This kind of methodology has been used successfully to isolate a peptide mimetic of erythropoietin (EPO) (Wrighton et al., 1996) with an apparent K_{d} of 0.2 μM. Examination of the three-dimensional structure of this complex with the EPO receptor (see Livnah et al., 1996) reveals that the peptide (20 residues) dimerizes to form a four-stranded anti-parallel β-sheet that is able to bind two EPO receptor molecules. However, the potency of this peptide is substantially lower than that of EPO. Although the peptide can induce levels of cell proliferation comparable to those induced by the cytokine, the concentration necessary to achieve a half maximal response, the EC₅₀, is 400 μM for the peptide and 20 pM for EPO. The response induced by the peptide in vivo was also found to be 100,000-fold lower than that of the protein.

A recently published study has reported the increase in the potency of this peptide through chemical covalent dimerization, so resolving the problem of inefficient dimerisation of the peptide (Wrighton et al., 1997).

A recombinant library was also used in the isolation of a 14-amino acid peptide that binds the thrombopoietin TPO) receptor with a 2 nM Kd and that stimulates cell proliferation with an EC50 of 400 nM (Cwirla et al.,1997). A covalent linkage strategy similar to the one described above has been used to produce a dimeric peptide with an EC50 of 100 pM that is equipotent with the cytokine.

A peptide antagonist of the human type I IL-1 receptor has also been discovered by screening recombinant peptide libraries (Yanofsky et al., 1996). The peptide blocks binding of IL-1α to the receptor with an inhibitory concentration (IC₅₀) of around 2 nM in human and monkey cell lines. Furthermore, the peptide shows high specificity for the human type I receptor and does not bind to human type II IL-1 receptor or to the murine type I receptor.

International patent application WO94/29332 (SmithKline Beecham Corporation) describes polypeptides containing typical antiparallel coiled-coils and teaches that these scaffolds can be adapted to form specific recognition molecules by the incorporation of helical recognition sequences from naturally-occurring proteins such as DNA binding proteins and cytokines. The α-helical structures used in the scaffolds comprise heptad repeats with a profile consisting of a hydrophilic exterior, a hydrophobic interior and a border of polar amino acid residues that form interhelical salt bridging residues. Positions in the heptad repeat show a strong preference for certain types of amino acid. The presence of these amino acid residues at these positions is important for the stability of the polypeptide. This method has not, to the inventors' knowledge, led to the development of biologically functional mimetics of cytokine molecules with potential as lead drug molecules.

Therefore, it remains of crucial importance both to devise novel strategies that allow the more efficient production of cytokine antagonists, and to generate alternative methods to block the interaction between cytokines and their receptors. Ideally, it would be desirable to design small molecules that are able to compete with cytokine for binding to its specific receptor.

### SUMMARY OF THE INVENTION

According to the invention there is provided a peptide mimetic of a cytokine molecule, said mimetic comprising an atypical helix-turn-helix motif mutated to incorporate one or more amino acid residues from the cytokine molecule.

Peptide mimetics according to the invention are stable by virtue of structural features inherent in the atypical helix-turn-helix motif, yet incorporate amino acid residues from a cytokine molecule such that the mimetics bind to targets of the cytokine in question with a high affinity and high specificity.

By “atypical” helix-turn-helix is meant a helix-turn-helix motif that does not conform to the structural features of proteins that contain “typical” helix-turn-helix (also known as coiled coil) motifs as these are defined below. The sequences of typical helix-turn-helix motifs are characterized by a repeating heptad of amino acids, (abcdefg)n.

The heptad is a repeating structural unit where the residues are distributed over two helical turns and every seventh amino acid is at a structurally equivalent position. The residues in a heptad (abcdefg) pack against the heptad on the opposite helix (a′b′c′d′e′f g′) according to the knobs-into-holes model described by Crick (Crick et al., 1953). As shown in FIG. 1, the local geometry of the packing is different for parallel and antiparallel helix-turn-helix motifs (Monera et al., 1994). If the two helices are parallel, residues at position d in one helix pack against the equivalent residue at position d′ in the other helix, and residues at position g interact with residues at position e′. On the other hand, if the helices have an antiparallel orientation, residues in position d pack against residues at position a′, and residues at position g interact with residues at position g′.

In typical helix-turn-helix motifs, positions in the heptad repeat show a strong preference for certain types of amino acids. Three main groups can be distinguished:

1. Positions a and d which make up the bulk layer of the hydrophobic interface are occupied by hydrophobic amino acids such as alanine, leucine, valine and isoleucine. Leucine is the amino acid found most frequently at position d whereas β-branched amino acids, like valine or isoleucine, are more common at position a. The preference of different residues for these positions is related to the local geometry of the packing (Betz et al., 1995).

2. Polar residues, especially those with charged side-chains, like lysine, arginine, glutamate and aspartate, are usually found at positions e and g. These residues are responsible for intra- and inter-helical electrostatic interactions and the aliphatic portion of the side chain contributes to the packing of the hydrophobic core.

3. Positions b, c and f are solvent-exposed and are occupied by hydrophilic residues. The side chains of residues at positions b and c might be involved in inter-helical electrostatic interactions with residues at positions e and g, respectively.

In contrast, the sequence of an atypical helix-turn-helix does not conform to the above rules. Accordingly, examination of the amino acid sequence alone is not generally predictive of the presence of a helix-turn-helix structure at any point in the molecule and examination of the structure of the molecule may be the only clue as to the presence of such a motif. Examples of proteins containing atypical helix-turn-helix motifs are ROP (GenBank accession no. P03051; wild type sequence given in SEQ ID NO:3); the dimerization domain of the Escherichia coli gene regulatory protein AraC (pdb code 2ara and 2aac) (Soisson et al., 1997a; Soisson et al., 1997b); the coiled-coil finger of the effector domain of protein kinase PKN/PRK1, known as the ACC finger, which is involved in binding to G protein Rhoa (pdb code 1 cxz) (Maesaki et al., 1999); and the coiled-coil of Thermus Thermophilus seryl-tRNA synthetase (pdb code 1ser) (Biou et al., 1994).

The atypical helix-turn-helix motifs in the proteins mentioned above are all extremely stable molecules, as a result of the inherent properties conferred by the encoding amino acid sequence. Accordingly, one principle embodied in the present invention is that the stability of the wild type molecule may be significantly compromised by the modification of the sequence to incorporate amino acid residues from a cytokine molecule, whilst still resulting in a stable peptide mimetic. This considerably facilitates the inclusion of a desired epitope into the scaffold helix-turn-helix, since the majority or entirety of the epitope may be grafted onto the scaffold sequence. It is thus not necessary to meet the stringent consensus requirements of typical helix-turn-helix motifs in order for the molecule to retain its stability. The invention thus has significant advantages over work previously described, for example, by SmithKline Beecham (WO94/29332).

The present invention is particularly suited to the design of mimetics of molecules such as cytokines, that possess discontinuous epitopes.

The helix-turn-helix of the ROP protein is a particularly preferred atypical helix-turn-helix motif for use in accordance with the present invention. ROP is an E. coli transcription factor that regulates the copy number of ColE1-related plasmids (Cesareni et al., 1982; Twigg & Sherratt et al., 1980). The sequence of the full length protein comprises 63 amino acids and the three-dimensional structure (Banner et al., 1987) indicates that it forms a helix-turn-helix motif that dimerizes in solution (see FIG. 2). In vivo, the functional protein contains two polypeptide chains that pack against each other. The resulting protein is very. stable to temperature and to chemically-induced denaturation, with a Tm of 64° C. and a concentration of guanidinium hydrochloride at the midpoint of the denaturation transition (Cm) of 3.3 M (Munson et al., 1996).

The helix-turn-helix of the ROP monomer forms an antiparallel coiled-coil. The inventors have noted that the structure of this monomer can be superimposed upon the structures of a variety of different cytokines, particularly the structures of four helix bundle cytokines such as interleukin 4 (IL-4), interleukin 2 (IL-2) and the human growth hormone (HGH). For example, the antiparallel coiled-coil of the ROP monomer can be superimposed on helices A and C of IL-4 (which bear the epitope for IL-4 receptor alpha) with a rmsd (root mean square deviation) of 1.2 Å for the Cα atoms (see FIG. 3).

As the skilled reader will be aware, it is not essential to use the full length, wild type sequence of the atypical helix-turn-helix. Variants of this sequence, including sequences that contain one or more amino acid insertions, deletions, or substitutions from the wild type sequence of the motif, may be applicable to the present invention.

For example, some parts of the helix-turn-helix sequence may not be necessary for inclusion in the peptide mimetic. It is preferred to discard elements of the full length sequence that have no positive effect either on the stability of the protein or on the protein conformation that is desired to present the cytokine epitope in the required configuration, since smaller peptides generally possess more desirable pharmacokinetic properties and are easier to produce, both by recombinant and by synthetic means. In particular, residues from the N terminus and/or the C terminus of the helix-turn-helix may generally be deleted without compromising the binding function.

Other residues may also be replaced or deleted, for example to aid in the expression of the peptide mimetic in preferred host species, to facilitate cloning of the molecule, to increase the stability of the peptide; to increase helix packing and so on. One example is the inclusion of a methionine residue at the N terminus of the peptide mimetic, to improve the efficiency of expression in bacterial hosts.

In the exemplary case of a peptide mimetic of IL-4 grafted onto the ROP atypical helix-turn-helix, the first and last seven terminal residues of the ROP sequence may preferably be deleted, because in the best alignment obtained between IL-4 and ROP, these residues extend beyond the positions of interest (see FIG. 3).

According to the present invention, a preferred mimetic may be constructed of any cytokine molecule, including interleukins, interferons, colony stimulating factors, tumour necrosis factors, growth factors and chemokines.

Preferred cytokines for which peptide mimetics may be constructed according to the invention are “four helix bundle” cytokines that belong to the haematopoietic or class I cytokine superfamily. The three-dimensional structure of these proteins consists of a four-helix bundle with an “up-up-down-down” topology, including between one to three disulphide bridges.

Based on the length of the polypeptide chain and on structural features, two main subfamilies may be identified in this superfamily. In addition, the interferons are frequently considered to constitute a third subfamily of the four helix bundle cytokines.

Members of this superfamily include the human growth hormone (HGH), granulocyte Macrophage-Colony stimulating factor (GM-CSF), granulocyte Colony stimulating factor (G-CSF), leukaemia inhibitory factor (LIF), erythropoietin (EPO), IL-2, IL-3, IL-4, IL-5, IL-6, IL-7, IL-9, IL-13, ciliary neurotrophic factor (CNTF), oncostatin (OSM) and the interferons among others. Some of the known members of these three subfamilies that have structures similar to IL-4 are listed in Table I below. Preferably, peptide mimetics of the invention mimic the short chain members of the Class I hematopoietic superfamily, or, from the long chain members, Growth Hormone. Even more preferably, peptide mimetics according to the invention mimic the cytokines IL-4, IL-2 or HGH, particularly IL-4.

The four helix bundle cytokines bind to a class of receptors known as the hematopoietin receptor superfamily or type 1 cytokine receptor superfamily. These receptors comprise an extracellular cytokine binding domain that is highly homologous within the family, a single transmembrane domain, and an intracellular domain that lacks intrinsic tyrosine kinase activity. The extracellular domain has four conserved cysteines and a characteristic tryptophan-serine-X-tryptophan-serine (the so-called tryptophan box) motif that is thought to be important for efficient receptor folding (for reviews see Bazan, 1990 and Gullberg et al 1995).

A striking feature of the haematopoietic cytokine superfamily is that, despite their common form, the family members have little or no sequence homology. However, the fact that these proteins are all extracellular signalling molecules, that they share the same unique four helix bundle topology and a similar gene organisation, suggests that they are all related by a process of divergent evolution.

TABLE I
Class 1 hematopoletic cytokine superfamily

| | Examples with known | |
|---|---|---|
| Subclass | structures | Putative Members |
| Short chain | IL-2, IL-4, IL-5 | IL-3, IL-7, IL-9 |
| | GM-CSF, M-CSFa | IL-13, IL-15 |
| Long chain | GH, LIF, G-CSF | OSM, IL-11, TPO |
| | IL-6 | IL-12, CNTF, PRL |
| | | EPO |
| Interferon | IFN-β and IFN-γ | |

CNTF, ciliary neurotrophic factor;
EPO, erythropoietin;
OSM, oncostatin;
TPO, thrombopoietin;
LIE, Leukemia inhibitory factor;
PRL, prolactin;
IFN, interferon.
*The protein is a non-covalent dimer; aThe protein is a disulphide linked dimer. This table is a modified and updated version of that in (Mott et al., 1995)

The most extensively characterised cytokine-receptor system is that of the human growth hormone. A determination of the three dimensional structure of this protein bound to two chains of the same receptor (de Vos et al 1992) has laid the grounds for understanding the principles that are involved in molecular recognition and signal transduction by four helix bundle cytokines and their receptors. From the data that are available in the literature on other cytokine receptor systems, it is clear that the ligand induced receptor homo- or hetero-oligomerisation is a general strategy that is used by members of the haematopoietic cytokine family.

By the term “peptide” is meant any short chain of amino acids (peptides and oligopeptides) comprising amino acids joined to each other by peptide bonds or by modified peptide bonds, i.e., peptide isosteres. Peptides according to the invention will generally be between 20 amino acids and 100 amino acids in length, preferably between 30 and 75 amino acids, more preferably between 40 and 60 amino acids in length.

The term “mimetic” means that the peptide mimics the binding site for the specific receptor of the cytokine. A cytokine's specific receptor is the receptor for which it exhibits the highest binding affinity. For example, in the case of IL-4, the specific receptor is lL-4Rα.

In order to be effective as a mimetic, peptides according to the invention should possess a high binding affinity for target. By “target” is meant any molecule that binds specifically to the cytokine in question with high affinity. Examples of targets include molecules that are necessary for the generation of a biological response in vivo; cytokine receptors are particularly preferred targets. For example, IL-4 is known to bind to a distinct receptor called the type II IL-4 receptor. In the case of IL-2, any of the IL-2 receptor chains are preferred targets. Similarly, the human growth hormone receptor is a preferred target for HGH.

The peptide mimetics of the invention should ideally be specific for target molecules of the wild type cytokine molecule. By this is meant that the peptide mimetic should ideally bind to target molecules with a similar affinity to the affinity with which the wild type cytokine binds, but also should not to any significant degree bind to molecules that the wild type cytokine molecule does not bind to. Of course, by careful screening, peptide mimetics according to the invention may be chosen to possess selected properties of the wild type cytokine molecule, to suit the application of choice (for example, binding to a subset of receptor targets bound by wild type cytokine).

In order to be useful in providing potential lead drug compounds, peptide mimetics of the invention should bind to target with an affinity of at least 1 mM, preferably 100 μM, more preferably, at least 50 μM, more preferably, at least 1 μM, more preferably, at least 100 nM, more preferably, at least 1 nM, most preferably, 100 pM or less.

The peptide mimetics of the invention may be incorporated as elements of fusion proteins. For example, it may be advantageous to include in one single protein a peptide mimetic according to the invention in conjunction with one or more amino acid sequences additional to the amino acids that are derived from the ROP protein or from the cytokine in question. Such amino acid sequences may contain secretory or leader sequences, pro-sequences, sequences which aid detection, expression, separation or purification, or sequences that confer increased protein stability, for example, during recombinant production. Such sequences may be fused at the amino- or carboxy-terminus of the modified cytokines. Examples of potential fusion partners include beta-galactosidase, glutathione-S-transferase, luciferase, a polyhistidine tag, a T7 polymerase fragment, a secretion signal peptide or another cytokine or cytokine receptor. Such derivatives may be prepared in any suitable manner, including by fusing the peptides genetically or chemically.

Peptide mimetics may also contain amino acids other than the 20 nucleotide-encoded amino acids, modified either by natural processes, such as by post-translational processing, or by chemical modification techniques which are well known in the art. The inclusion of such amino acids may resolve a problem that is inherent in the pharmaceutical use of linear natural peptides, which are generally degraded and/or eliminated rapidly in vivo.

Examples of known modifications which may commonly be present in polypeptides of the present invention are glycosylation, lipid attachment, sulphation, gamma-carboxylation of glutamic acid residues, hydroxylation and ADP-ribosylation, for instance. Other potential modifications include acetylation, acylation, ADP-ribosylation, amidation, covalent attachment of flavin, covalent attachment of a haeme moiety, covalent attachment of a nucleotide or nucleotide derivative, covalent attachment of a lipid or lipid derivative, covalent attachment of phosphotidylinositol, cross-linking, cyclization, disulphide bond formation, demethylation, formation of covalent cross-links, formation of cysteine, formation of pyroglutamate, formylation, gamma-carboxylation, glycosylation, GPI anchor formation, hydroxylation, iodination, methylation. myristoylation, oxidation, proteolytic processing, phosphorylation, prenylation, racemization, selenoylation, sulphation, transfer-RNA mediated addition of amino acids to proteins such as arginylation, and ubiquitination.

Modifications can occur anywhere in the peptide, including in the peptide backbone, the amino acid side-chains and the amino or carboxyl termini. In fact, blockage of the amino or carboxyl group in a peptide, or both, by a covalent modification, is common in naturally-occurring and synthetic peptides and such modifications may also be present in peptides of the present invention.

The modifications that occur may be a function of how the polypeptide is made. For polypeptides that are made recombinantly, the nature and extent of the modifications will in large part be determined by the post-translational modification capacity of the particular host cell and the modification signals that are present in the amino acid sequence of the polypeptide in question. For instance, glycosylation patterns vary between different types of host cells.

A particularly preferred peptide mimetic according to the present invention is a mimetic of IL-4. Interleukin 4 (IL-4) is a multifunctional cytokine produced mainly by T helper lymphocytes type 2 (TH2), which is involved in the regulation of different biological processes (for a review see Paul et al., 1991). This cytokine controls the growth and differentiation of various types of immune cells, and is involved in defence against helminthic macroparasites, and in the rejection of certain tumours. Perhaps the most important clinical role of IL-4 is the specific induction of Immunoglobulin class switching of B-cells expressing IgM into IgG4 and IgE (De Vries et al., 1991) and the up-regulation of the expression of the IgE low affinity receptor (CD23) on mast cells and B cells (Conrad et al., 1987). It is now evident that IL-4 plays a dominant role in the allergic response, as this protein determines whether B-cells give rise to IgE or to other types of antibodies. Consequently, drugs that are able to interfere with the activity of IL-4 will help reduce IgE levels and will render allergic reactions amenable to pharmaceutical control.

The mechanism of action of IL-4 at the surface of target cells, is thought to be by first binding to the IL-4 alpha chain receptor (IL-4Rα), with a K_{d} of 100 pM, so forming a binary complex that then recruits a second receptor component, which can be either the interleukin 2 (IL-2) receptor γc (Russel et al., 1993) chain or the interleukin 13 (IL-13) receptor a subunit (IL-13Rα), depending on the type of cell (for a review see Chomarat & Banchereau et al., 1998).

An example of a peptide mimetic of IL-4 is the peptide referred to herein as solup10; this peptide forms a preferred aspect of the invention. The sequence of this peptide is provided as SEQ ID NO:1. This molecule comprises the atypical helix-turn-helix motif of ROP modified to incorporate amino acid residues from the epitope of IL-4.

Functionally-equivalent variants of this peptide are intended to be included within this aspect of the invention, provided that such variants are effective to block binding of the IL-4 molecule to the IL-4 receptor. By the term “functionally-equivalent” is meant that the variant peptide mimetics of the invention inhibit one or more of the biological functions possessed by the wild type cytokine. For example, in the case of IL-4, such functions include the control of the growth and differentiation of immune cells, defence against helminthic macroparasites, the rejection of certain tumours and the specific induction of IgE antibodies.

Biological functions of other cytokines will be clear to those of skill in the art.

Functionally-equivalent variants may be, for example, mutants of the ROP or cytokine sequence containing amino acid substitutions, insertions or deletions, as well as natural biological variants (e.g. allelic variants or geographical variations within the species from which the cytokine molecule is derived). This term also refers to molecules that are structurally similar to the wild type cytokine, or that contain similar or identical tertiary structure. Variants with improved function from that of the wild type sequence may be designed through the systematic or directed mutation of specific residues in the protein sequence.

According to a further aspect of the invention, there is provided a method for the generation of a peptide mimetic of a cytokine molecule, said method comprising grafting the epitope of a cytokine molecule into the sequence of an atypical helix-turn-helix motif.

When designing peptide mimetics according to the method of this aspect of the invention, the cytokine binding site should first be identified. Elements from this moiety must be grafted onto the helix-turn-helix scaffold in order to ensure that the resulting peptide possesses the desired cytokine properties. Methods for the identification of the cytokine binding site will be clear to those of skill in the art; in most cases, the binding site will be known from published literature. For example, in the case of the four helix bundle cytokine IL-4, the required epitope comprises amino acid residues Ile5, Glu9, Thr13, Lys77, Arg81, Lys84, Arg85, Arg88, Asn89 and Trp99 of the IL-4 sequence.

In cases where the epitope is not known, the epitope may be identified using known methods of random or site-directed mutagenesis, followed by assaying for function.

The cytokine epitope may preferably be transferred to the surface of the helix-turn-helix at the dimer interface. In this fashion, the hydrophobic interface of the helix-turn-helix monomer may be disrupted in order to prevent the formation of peptide mimetic dimers. Once the epitope is grafted onto the monomer, the peptide obtained by this process should recognise and bind to the respective cytokine receptor, as required. This resulting monomer should also show significant stability to thermal and chemical denaturation. Preferably, the Tm and Cm of peptide mimetics produced according to the method of this aspect of the invention is as high as possible.

The method of this aspect of the invention preferably comprises an additional step of assaying a peptide mimetic, generated by grafting the epitope of the cytokine molecule into the sequence of an atypical helix-turn-helix, for functional activity, and mutating the sequence of the peptide to improve its cytokine activity. Preferably, the steps of design, assay and mutation are applied iteratively to increase the chances of obtaining a peptide mimetic with the desired properties.

Methods for assaying for functional activity may utilise binding assays, such as the enzyme-linked immunosorbent assay (ELISA), radioimmunoassay (RIA), fluorescence activated cell sorting (FACS) and other methods that are well known in the art (see Hampton, R. et al. (1990; Serological Methods a Laboratory Manual, APS Press, St Paul, Minn.) and Maddox, D. E. et al. (1983; J. Exp. Med. 158:1211-1216). Alternatively, assays may test the ability of the peptide mimetic in eliciting a biological response as a result of binding to a biological target, either in vivo or in vitro. Such assays include B cell and T cell proliferation assays, and inhibition of proliferation assays (see Paul et al., (1991). Other suitable assays will be known to those of skill in the art.

The step of mutation may be random or may be rational. Methods for the random mutation of peptide sequences will be known to those of skill in the art and include the use of phage display libraries to allow the biopanning of recombinant peptides, to screen for phage expressing peptide mimetics with high affinities for target molecules, for example cytokine receptors. Alternatively, a eukaryotic display library system can be used (for example, an insect cell library), based on the expression of foreign cDNAs on the surface of a virus such as a baculovirus (see, for example, Davies (1995) Bio/Technology 13, 1046; Boublik et al., (1995) Bio/Technology 13, 1079-1084). A phagemid vector system such as that described by Otto (1986) is also well-suited for this approach, although other systems will be readily apparent to the skilled reader (see Bradbury, 2000, for review).

The mutation process may be applied to all amino acid positions in the sequence of the peptide initially generated or, more preferably, will be applied selectively to specific amino acid positions that are known as being influential either for activity or stability.

The steps of a preferred method of the invention may be summarised as follows: i) a first molecule that binds the target with low affinity is designed rationally, ii) a few positions are then randomised to screen for stronger affinities; iii) the hits obtained during the first screens of random selection can be identified and their biophysical properties determined; iv) this information, together with other available functional data, may then be used to select the most suitable positions for further substitutions as well as to restrict the group of compounds to be included in the libraries.

The main disadvantage of screening methods is the long time necessary to pan the libraries for binding to the target. It is then necessary to characterise the best hit and start another time-consuming round of selection. A precious amount of time can thus be spared by applying rational design approaches at different stages of this process. It is, therefore, important to devise rational strategies that integrate structural and mutagenesis data. Molecular dynamic (MD) simulations provide one method that can assist rational design in selecting the most promising candidates in terns of foldability (see Cregut et al., 1999). One alternative is the algorithm for automatic protein design termed PERLA (see co-owned U.S. patent application Ser. No. 09/387,741). This algorithm can be used to aid the discovery of mutations that are candidates for increasing the binding affinity of a protein for a target ligand domain.

Other computational methods will be clear to those of skill in the art. Molecular mimics designed in this way are likely to provide reliable starting points with affinities comparable to those found in the first rounds of combinatorial screening studies (typically in the high μM range).

Such an approach combining phage display and rational design has previously been used to improve the stability and affinity of a two-helix derivative of the three-helix Z-domain of protein A. This 59 residue three-helix bundle binds the Fc portion of immunoglobulin G (IgG) with a Kd of 10 nM. The binding domain has been reduced to a 33 residue peptide that is able to bind IgG with virtually the same affinity as the wild-type protein (Braisted et al., 1996).

The peptide mimetics generated by the methods of this aspect of the invention may be used for the purification of target receptor. Accordingly, the invention provides a method for the preparation of a cytokine receptor comprising passing a composition containing the cytokine receptor over a matrix to which a peptide mimetic according to the invention is bound. The peptide mimetic may be immobilised on any suitable matrix, such as an affinity column, or a preparation of beads, such as sepharose beads.

In the case of IL-4 mimetics, a suitable target receptor for purification is Il-4Ralpha. Presently, IL-4Ralpha is purified by affinity chromatography on extremely expensive purification columns that are packed with wild type IL-4. The peptide mimetics generated by a method as described above will be much less expensive to produce in large quantities and will therefore allow purification columns to be produced at a lower cost, so diminishing the cost of preparing IL-4Ralpha protein for therapeutic use. Accordingly, the invention provides a method for the preparation of IL-4Ralpha protein comprising passing a composition containing IL-4Ralpha through an affinity column to which a peptide mimetic of the invention is bound, washing the column, and eluting IL-4Ralpha from the column.

According to a still further aspect of the present invention, there is provided the use of a peptide mimetic of a cytokine according to any one of the aspects of the invention described above to produce antibodies against the cytokine. In particular, an IL-4 mimetic as described above may be used in this aspect of the invention. Antibodies generated by this method have important uses as diagnostic and therapeutic tools, particularly when used in conjunction with the peptide mimetics of the invention.

According to a further aspect of the invention there is provided a nucleic acid molecule encoding a peptide mimetic according to any one of the aspects of the invention described above. One particularly preferred embodiment of this aspect of the invention comprises a nucleotide sequence encoding solup10 (amino acid sequence given in SEQ ID NO:1). In a preferred embodiment, this sequence may comprise or consist of the nucleotide sequence given in SEQ ID NO: 2. The invention also includes a vector containing such a nucleic acid molecule.

A further aspect of the invention provide a method for the production of a peptide mimetic as described above, comprising introducing a nucleic acid encoding the peptide mimetic into a host cell, such as an E. coli bacterium.

According to a still further aspect of the invention, there is provided a peptide mimetic according to any one of the above-described aspects of the invention, for use as a pharmaceutical. A further aspect of the invention provides for the use of such peptide mimetics in the manufacture of a medicament for the treatment or prevention of a disease in a mammal, preferably a human. Advantageously, the disease may be an allergy-related condition. The invention also provides a method of preventing or treating an allergy comprising administering to a patient a peptide mimetic as described above.

According to a still further aspect of the invention, there is provided a pharmaceutical composition comprising a peptide mimetic according to any one of the above-described aspects of the invention, optionally as a pharmaceutically-acceptable salt, in combination with a pharmaceutically-acceptable carrier. The invention also provides a process for preparing such a pharmaceutical composition, in which such a peptide mimetic is brought into association with a pharmaceutically-acceptable carrier.

According to a still further aspect of the invention, there is provided a diagnostic kit comprising a peptide mimetic according to any one of the above-described aspects of the invention. Such kits allow the detection of a biological target of the cytokine in question, such as a cytokine receptor, and are thus useful in the diagnosis and prognosis of diseases in which the particular cytokine or cytokine receptor is implicated. Furthermore, in certain conditions in which a cytokine receptor is overexpressed, the relative success or failure of a therapeutic treatment of the condition may be assessed by following the levels of a biological target over time.

The invention also provides a transgenic non-human mammal, carrying a transgene encoding a peptide mimetic according to any one of the above-described aspects of the invention. A further aspect of the invention provides a process for producing such a transgenic animal, comprising the step of introducing a nucleic acid molecule encoding the peptide mimetic into an embryo of a non-human mammal, preferably a mouse.

Various aspects and embodiments of the present invention will now be described in more detail by way of example, with particular reference to peptide mimetics of IL-4. It will be appreciated that modification of detail may be made without departing from the scope of the invention.

### BRIEF DESCRIPTION OF THE FIGURES

FIG. 1: Helical wheel representation of a prototypic heptad in a parallel and antiparallel coiled-coil. Note the difference in the packing of the core, and the establishment of intra and inter-chain electrostatic interactions between distinct heptad positions. Adapted from Monera et al., 1994.

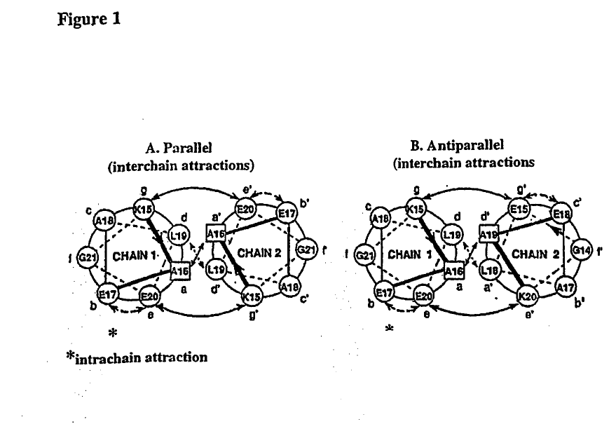

FIG. 2: Ribbon representation of the ROP protein (pdb code 1rop).

FIG. 3: Superimposition of the ROP coiled-coil (black) on helices A and C of IL-4 (grey).

FIG. 4: Stereo view of the superimposition of the energy-minimized model of solup10 on helix A and C of IL-4, showing the side chains involved in binding to ILA4Rα.

FIG. 5: Far UV CD spectra of solup10 at 5° C. before and after temperature denaturation. The fact that the signal can be completely recovered shows that the denaturation process is reversible.

FIG. 6: Temperature-induced denaturation profile of solup10.

FIG. 7: Coiled-coil domain of the seryl-t RNA synthetase (Biou et al., 1994) superimposed on helices A and C of IL-4. The rmsd deviation for the backbone Cα atoms is 1.3 Å. It is possible to shift the alignment in order to look for the more convenient positions to mutate.

### EXAMPLES

### Example 1

Rationale for Molecule Design

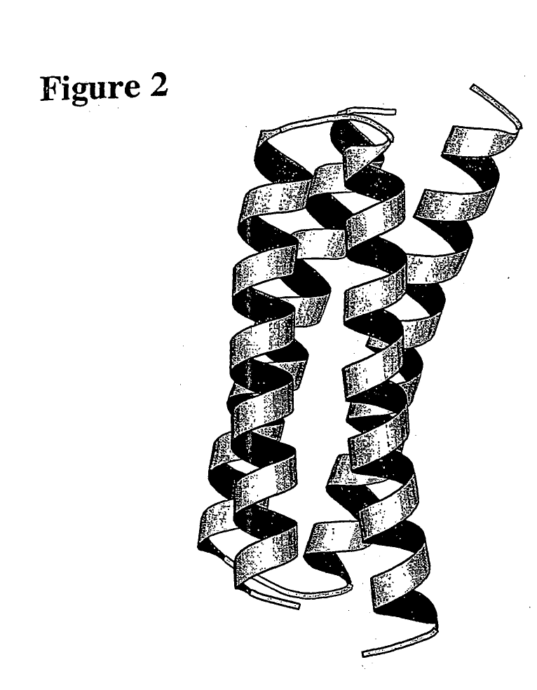

ROP is an E. coli transcription factor that regulates the copy number of ColE1 related plasmids (Cesareni et al., 1982; Twigg & Sherratt et al., 1980). The sequence of the protein comprises 63 amino acids and the three-dimensional structure (Banner et al., 1987) shows that they form a helix-turn-helix motif that dimerizes in solution (FIG. 2). The functional protein contains two polypeptide chains that pack against each other and is very stable to temperature or chemically induced denaturation, with a Tm of 64° C. and a Cm (concentration of guanidinium hydrochloride at the midpoint of the denaturation transition) of 3.3 M (Munson et al., 1996).

The structural features shared in common between typical helix-turn-helix motifs are discussed in some detail above. As is apparent from the primary sequence of ROP, the coiled-coil is atypical for a number of reasons. These are:

1. Several polar and charged residues are found in position a—Glu5, Thr19, Asp59, Cys38, Cys52, and d—Gln34, Arg55, Asn62.

2. Hydrophobic residues are found at position e—Leu9, Leu23, Ala35, Tyr49, Phe56, Leu63, and g—Met12, Ile37.

3. Hydrophobic residues like Leu20, Leu53 and Phe14, Ala20, also occupy, respectively b and c positions.

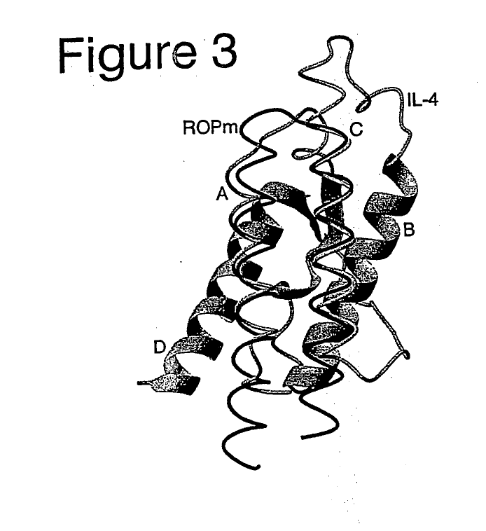

FIG. 3 represents the structural alignment of the ROP monomer with helix A and C of IL-4; Table 1 shows the corresponding sequence alignment.

Table 1 Alignment of the ROP sequence with the assignment of heptad positions and the sequence of helix A and C of IL-4.

| 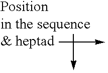 | f | g | a | b | c | d | e |
|---|---|---|---|---|---|---|---|
| 1 | Met | Thr | | | | | |
| 3 | Lys | Gln | Glu | Lys | Thr | Ala | Leu |
| 10 | Asn | Met | Ala | Arg | Phe | Ile | Arg |
| IL-4 Helix A | | [symbol] | Thr | Leu | Gln | [symbol] | ¹⁰ Ile |
| 17 | Ser | Gln | Thr | Leu | Thr | Leu | Leu |
| | ¹¹Ile | Lys | [symbol] | Leu | Asn | Ser | ¹⁷Leu |
| 24 | Glu | Lys | Leu | Asn | Glu | Leu | Asp |
| | ¹⁸Thr | Glu | ²⁰Gln | | | | |
| 31 | [symbol] Ala → | | | Asp | Glu | Gln | Ala |
| IL-4 Helix C | | | | | [symbol] | Gln | ⁷⁹ Leu |
| 36 | Asp | Ile | Cys | Glu | Ser | Leu | His |
| | ⁸⁰Ile | [symbol] | Phe | Leu | [symbol] | [symbol] | ⁸⁶Leu |
| 43 | Asp | His | Ala | Asp | Glu | Leu | Tyr |
| | ⁸⁷Asp | [symbol] | [symbol] | Leu | [symbol] | ⁹²Gly | |
| 50 | Arg | Ser | Cys | Leu | Ala | Arg | Phe |
| 57 | (Gly | Asp | Asp | Gly | Glu | Asn | Leu) |
| ROP residues 1 and 2 are non-helical and the turn occurs around alanine 31, interrupting the heptad pattern. From Gln34 on (the second helix of the coil), the nomenclature of the heptad should be taken as (a′b′c′d′e′f′). Residues 57 to 63 are not visible in the X-ray structure. Residues of the IL-4 functional epitope are underlined and in italic. Residues that are the same | | | | | | | |
| # in ROP and IL-4 are shown in bold. | | | | | | | |

In view of the above, it is evident that ROP does not exhibit the standard helix-turn-helix (coiled-coil) characteristics. In fact, from the sequence alone, the protein would hardly be predicted to fold as a coiled-coil. The protein does, however, show the typical coiled-coil fold. The rationale for the proposal that ROP might be a suitable scaffold to design an IL-4 mimetic is as follows:

a) The helices show the same backbone orientation as helices A and C of IL-4.

b) It is possible to superimpose the backbone Cα atoms of both proteins with a small rmsd (1.2 Å).

c) The structural alignment reveals that the same residues are found at some positions in both proteins (shown in bold in table 1). One of these residues (Thr13) is part of the IL-4 functional epitope and, because it aligns with Thr19 in ROP, it was not necessary to mutate this residue in the ROP sequence. The fact that some amino acids at the aligned positions are the same in ROP and IL-4 might also contribute to reduce the immunogenicity of a ROP-derived IL-4 antagonist.

### Example 2

Grafting the IL-4 Epitope into the ROP Monomer

The IL-4 epitope was transferred to the surface of the ROP coiled-coil, at the dimer interface. The goal was to disrupt the hydrophobic interface in order to prevent the formation of dimers. The monomer thus obtained should recognize and bind IL-4Rα.

The first and last seven terminal residues of the ROP sequence were deleted because in the best alignment obtained, they extend beyond the positions of interest (see FIG. 3). Alanine 8 was replaced by methionine in order to allow overexpression of the protein in prokaryotic hosts. A Glycine residue was introduced after the methionine to allow cloning into the NcoI site. A N-terminal helix capping was designed by mutating Leu9 into threonine and Ala12 into glutamine. Asparagine 10 was replaced by a negatively charged residue (Asp) in order to establish a favourable interaction with the helix macrodipole.

The IL-4 binding site for IL-4Rα was introduced at the corresponding positions shown in table 1. The following mutations were designed in order to stabilize the ROP-derived IL-4 mimetic: Ser17, Thr21, Asp43 were replace by alanine to increase the helical propensity of the sequence. In order to improve the packing of the two helices Cys38, His42, were replaced by leucine. Glutamate 39 was replaced by arginine in order to form a salt bridge with Asp36. The packing of the termini of the helices was improved by replacing the bulky side chain of Tyr49 by that of phenylalanine. The helix was terminated by a glycine residue followed by a serine.

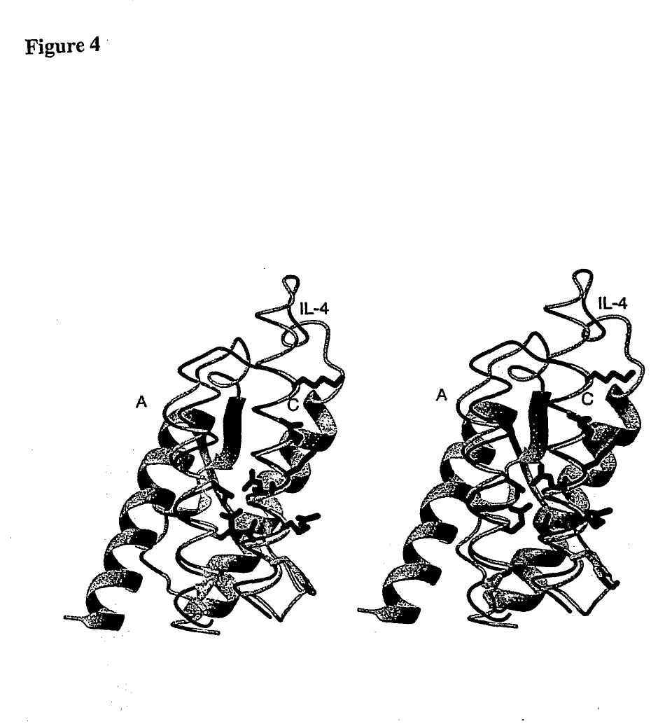

In this way, a 45-residue mini-protein that will be herein termed solup10 was obtained. The sequence of this protein is listed in table 2 together with the sequence of ROP used as a template. The model is shown in FIG. 4.

TABLE 2
Sequence of the ROP-derived protein containing the
IL-4 binding site and the original ROP sequence
used as a template.

ROP
¹ALNMARFIRSQTLTLLEKLNELDADEQADICESLHDHADELY⁴²
Solup10:
¹MGTDIQRYERAQTLALLEKLNELDADKQADRLRKRLARNDWLFGS⁴⁵
The IL-4 binding site is shown in bold and the residues mutated in order to increase the stability of the protein (see text) are in italic.

### Example 3

The Molecular Modeling Procedure

The crystallographic structure of the ROP protein from Banner et al. (Banner et al. 1987) (PDB entry: 1rop) was used as a template for the design. The sidechains of the mutated positions on the peptides were built using the program SMD (Tuffery et al., 1991) while maintaining fixed all other residues. This program uses a rotamer database to find the optimal rotamer at a given position of a 3D structure. Energy minimization using the AMBER force field (Cornell et al., 1995) including all atoms, as implemented in the AMBER 4.1 package (Pearlman et al., 1995).

The following strategy was followed: the mutated side chains were subjected to 500 cycles of minimization; then all the side chains were minimized during 1000 cycles; and finally all the system was minimized for 1000 cycles in order to reduce any bad contacts. All calculations were performed on a Silicon Graphics Octane/R10000 workstation.

### Example 4

Cloning, Expression and Purification of Solup10

Solup10 was cloned using a synthetic gene with the following sequence:

5′CAAGGCGCCATGGGTACCGACATCCAGCGTTACGAACGTGCTCAGACC
CTGGCACTGCTGGAAAAACTGAACGAACTGGACGCGGACAAACAGGCAGA
TCGCCTGCGCAAACGTCTGGCGCGAAACGACTGGCTGTTCGGTTCCTGAT
AAAGGTTATATAT3′

NcoI and HindIII restriction sites were introduced at the 5′ and 3′ends, respectively (underlined in the sequence above). The gene encoding the protein was cloned as a GST (glutathione-S-transferase) C-terminal fusion protein in PGAT2 (Peränen et al., 1996). The GST coding region is followed by a linker sequence containing the thrombin consensus site that consists of: Leu Val Pro Arg Gly Ser, With the thrombin cleavage occurring between the Arg and the Gly residues. The gene of solup10 was inserted after the linker.

Escherichia coli BL21 (DE3) from Novagen were transformed with the plasmid. 1 L flasks with LB medium were inoculated with a single colony and incubated on a shaker at 37° C. After an OD₆₀₀ of 0.7 was reached IPTG was added resulting in a final concentration of 0.16 mM. The culture was incubated for three hours and the cells harvested by centrifugation. The harvested cells were resuspended in PBS (Phosphate buffer saline). A tablet of a cocktail of protease inhibitors from Boehringer Mannheim (complete—EDTA free) and DNase to a final concentration of 10 μg/ml were added to the cell extract which was then incubated on ice for 30 minutes. Cell disruption was performed on a French Pressure cell. The soluble fraction containing the fusion protein was separated from the insoluble debris by ultracentrifugation at 40 000 rpm/min, at 4° C., for 1 hour. Triton was added to a final concentration of 1% followed by 30 minutes incubation at room temperature (RT). 2 ml of 50% slurry gluthathione Sepharose 4B (Pharmacia) were added to 40 ml of supernatant. After incubation at room temperature for 30 minutes the solution was transferred to a disposable column, and washed three times with 10 bed volumes of PBS, then with one bed volume of 10 mM TrisHCl pH 8.0/1M NaCl. The elution of the protein was carried out by adding 1 ml of glutathione elution buffer (10 mM reduced glutathione in 100 mM Tris HCl pH 8.5) per ml of bed volume, and incubating at RT for 10 minutes. The GST-solup10 fusion protein was digested with thrombin by adding 10 cleavage units of thrombin/mg of fusion protein. The cleaved Solup10 contained an additional glycine and serine residue at the N-terminal and was separated from the GST and the protease by FPLC purification on a superdex 75 column (Pharmacia) pre-equilibrated with PBS.

### Example 5

Properties of Solup10

Circular Dichroism

The far UV CD spectra of the peptide was recorded, on a Jasco-710 instrument, at 278 K, in a cuvette with a 2 mm path. Measurements were made every 0.1 nm, with a response time of 2 s and a bandwidth of 1 nm, at a scan speed of 50 nm/min. The spectra shown in the text represent an average over 20 scans. The peptide concentration, calculated from the absorbance at 280 nm (Pace et al., 1995) was 15 μM. The helical percentage was calculated from the mean residue ellipticity at 222 nm, taking into account the peptide length (Chen et al., 1974), according to the following:

%Helix=100 θ^{obs}_{222 nm}/(39 500(1−2.57/n)

where n is the number of residues in the peptide and θ^{obs}_{222 nm} is the ellipticity of the peptide at 222 nm. The CD experiments were carried out using the same buffer conditions as in the surface plasmon resonance assays.

Temperature Denaturation

The thermal denaturation was measured by monitoring the change in signal at 222 nm over a temperature range of 5-80° C., in a cuvette with a 2 mm path. Measurements were made in 0.5 degrees increments, with a response time of 2 s and a bandwidth of 1 nm, at a temperature slope of 50° C./h. The peptide concentration calculated from the absorbance at 280 nm was 15 μM (Pace et al., 1995).

Activity Assays

The binding affinities of the designed peptide mimetics were measured by surface plasmon resonance using a BIAcore 2000 (Pharmacia Biosensor AB). A recombinant extracellular domain of the receptor α-chain [C182A,Q206C]-IL4-BP was immobilized at a biosensor CM5 to a density of 1500 to 2000 pg/mm², as described by Shen et al. (Shen et al., 1996). Ligand binding was analyzed at 25° C. by perfusion with HBS buffer (10 mM Hepes, pH 7.4/150 mM NaCl/3.4 mM EDTA/0.005% surfactant P20) at a flow rate of 50 μl/min.

Toxicity

Experiments with HeLa cells have shown that the solup10 protein is non-toxic to mammalian cells at least up to a concentration of 500 μM, both alone and in combination with IL-4 at concentrations of up to 500 μM.

Results

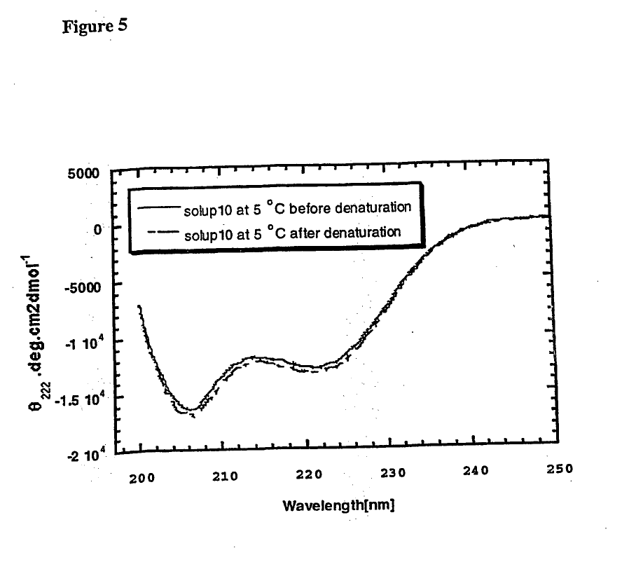

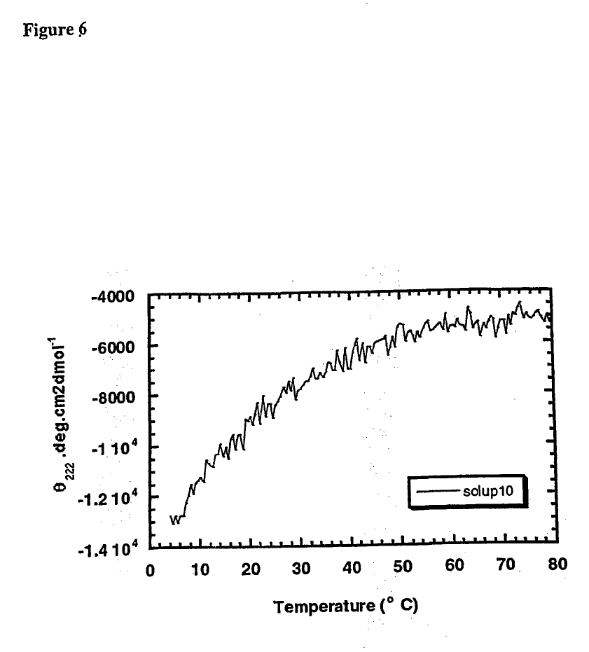

In order to investigate whether solup10 showed a helical conformation, as expected, the CD spectrum of the sample was measured in the far UV. As shown in FIG. 5 the spectrum displays a minimum at 207 nm and another at 222 nm which are characteristic of helical structure. A helical content of 34% was calculated as described above was. The temperature-induced denaturation (FIG. 6) is a reversible process with the CD signal being completely recovered upon cooling the sample back, from 80 to 5° C. (FIG. 5).

A surface plasmon resonance binding assay with IL-4Rα immobilized at the biosensor matrix showed that this new system binds the receptor with a 40 μM K_{d}, under physiological salt concentration. The binding assays were carried out a 25° C. and, as it can be seen in FIG. 6, at this temperature, solup10 has lost half of its helical content.

This observation suggests that the K_{d} can be improved by increasing further the structural stability of the molecule. This would make solup10 a more stable and stronger antagonist of IL-4. In practical terms this might translate into the need of lower and less frequent therapeutic doses.

The work presented here shows that it is possible to design rationally cytokine mimetics that have affinities comparable to those obtained in the first rounds of combinatorial screening. The molecule we have designed (solup10) might be used as an IL-4 antagonist but is also an excellent lead for further optimization by phage display. As stated previously, phage display has been used to obtain mimetics of three very important cytokines: erythropoietin (EPO), thrombopoietin and interleukin-1. EPO controls the proliferation and differentiation of immature erythroid cells (Jacobs et al., 1985), thrombopoietin regulates the development of platelet precursor cells (Foa et al., 1994), and IL-1 is involved in a number of autoimmune and inflammatory disorders. While thrombopoietin is still in clinical trials (Cwirla et al., 1997), recombinant erythropoietin is utilized in the treatment of anemia associated with certain disease states (Foa et al., 1994), and interleukin-l protein antagonists have showed promising therapeutic value in several animal models and clinical settings (Eastgate et al., 1990).

Like the vast majority of protein drugs, these cytokines must be administered by intravenous or subcutaneous injection. The discovery of mini-proteins that mimic the action of these hormones, and the observation that their activity and biostability can be optimized, has brought hope that such proteins may be developed into therapeutic molecules.

Like the cytokines referred to above, IL-4 is of very high therapeutic interest, particularly due to its important role in the pathology of allergic reactions. However, the therapeutic value of antagonist proteins generated to date (Kruse et al., 1992; Tony et al., 1994) is precluded by the fact that they form inclusion bodies when overexpressed in E. coli and refold in vitro with very low yields (van Kimmenade et al., 1988). Therefore, it is very important to develop alternative methods to avert the action of IL-4.

Mimetics of IL-4, which are of much smaller size and can be produced in large amounts are an attractive possibility, since these molecules can be used as lead compounds that can be further optimized through combinatorial screening and may be emulated by small organic frameworks that offer the biostability and bioavailability required for therapeutic drugs.

It should also be mentioned that molecules that bind to IL-4Rα are also potential antagonists of IL-13 because this cytokine requires IL-4Rα for signaling (Chomarat & Banchereau et al., 1998; Grunewald et al., 1998). IL-13, together IL-4 and IL-5, plays an important role in the pathophysiology of allergic reactions. Therefore, the ability to block IL-4Rα would allow the simultaneous inhibition of two key cytokines at the basis of allergic disease states.

Finally, IL-4 mimetics that bind IL-4Rα might also be used in the affinity purification of IL-4Rα (Hage et al., 1998) instead of IL-4 that is much more difficult to produce.

It is thought that the design strategy that we followed has an enormous potential in the design of cytokine mimetics, specially of type I cytokines (see table 3). Some of these proteins, including interleukin 2 (IL-2) and growth hormone (GH), display epitopes in two of the helices, like IL-4. Residues in helices A and C of IL-2 are engaged in the binding to IL-2Rβ (Zurawski et al., 1993), and site II of GH has also been mapped to helices A and C (De Vos et al., 1992). These cytokine-receptor systems are even more suitable to his kind of approach because the affinities are lower than in the case of IL-4/IL-4Rα (Fuh et al., 1992; Zurawski et al., 1993).

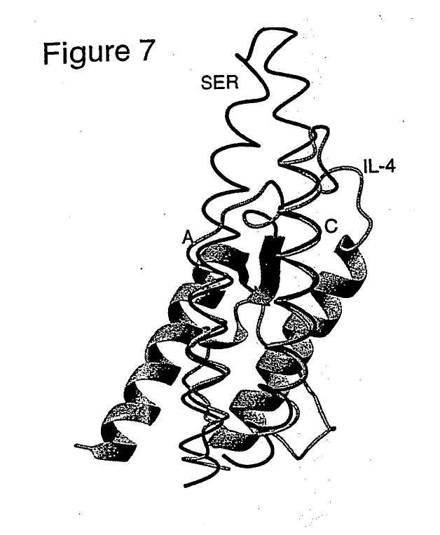

Finally, it should be stressed that the design strategy described here can also be applied to the cytokine-receptor systems referred to above, using antiparallel coiled-coil templates different from ROP. As stated previously, the coiled-coil is a rather promiscuous motif, particularly in structural and DNA binding proteins. Therefore, it is very likely that several of the dimeric or multimeric coiled-coils in the pdb can be engineered into cytokine mimetics. Some possible candidates include the dimerization domain of the Escherichia coli is gene regulatory protein AraC (pdb code 2ara and 2aac) (Soisson et al., 1997a; Soisson et al., 1997b); the coiled-coil finger of the effector domain of protein kinase PKN/PRK1, known as the ACC finger, which is involved in binding to G protein Rhoa (pdb code 1cxz) (Maesaki et al., 1999); and the coiled-coil of Thermus Thermophilus seryl-tRNA synthetase (pdb code 1ser) (Biou et al., 1994). The latter is shown in FIG. 7 superimposed on helices A and C of IL-4.

SEQUENCE LISTING

| | |
|---|---|
| (Solup10) | SEQ ID NO:1 |

MGTDIQRYERAQTLALLEKLNELDADKQADRLRKRLARNDWLFGS

| | |
|---|---|
| (Nucleic acid encoding Solup 10) | SEQ ID NO:2 |

CAAGGCGCCATGGGTACCGACATCCAGCGTTACGAACGTGCTCAGACCCT
GGCACTGCTGGAAAAACTGAACGAACTGGACGCGGACAAACAGGCAGATC
GCCTGCGCAAACGTCTGGCGCGAAACGACTGGCTGTTCGGTTCCTGATAA
AGCTTATATAT

| | |
|---|---|
| (ROP protein) | SEQ ID NO:3 |

MTKQEKTALNMARFIRSQTLTLLEKLNELDADEQADICESLHDHADELYR
SCLARFGDDGENL

References

Banner, D. W., Kokkinidis, M. & Tsernoglou, D. (1987). Structure of the ColE1 rop protein at 1.7 A resolution. J Mol Biol 196(3), 657-75.

Bazan, J. F. (1990). Structural design and molecular evolution of a cytokine receptor superfamily. Proc. Natl. Acad. Sci. USA 87, 6934-6938.

Betz, S. F., Bryson, J. W., DeGrado, W. F. (1995). Native-like and structurally characterized designed alpha-helical bundles. Curr. Opin. Struct. Biol. 5, 457-463.

Biou, V., Yaremchuk, A., Tukalo, M. & Cusack, S. (1994). The 2.9 A crystal structure of T. thermophilus seryl-tRNA synthetase complexed with tRNA(Ser). Science 263(5152), 1404-10.

Bradbury, A. (2000) Coupling phenotype to genotype: selecting phages, ribosomes and artificial cells. Tibtech 18, 3-5.

Braisted, A. C., Wells, J. (1996). Minimizing a binding domain from protein A. Proc. Natl. Acad. Sci. USA 93, 5688-5692.

Buckel, P. (1996). Recombinant proteins for therapy. Trends in Protein Science 17, 450-456.

Cesareni, G., Muesing, M. A. & Polisky, B. (1982). Control of ColE1 DNA replication: the ROP gene product negatively affects transcription from the replication primer promoter. Proc Natl Acad Sci USA 79(20), 6313-7.

Chen, Y.-H., Yang, J. T., Chau, K., H. (1974). Determination of the helix and b-form of protein in aqueous solution by Circular Dichroism. Biochemistry 13(16), 3350-3359.

Chomarat, P. & Banchereau, J. (1998). Interleukin-4 and interleukin-13: their similarities and discrepancies. Int Rev Immunol 17(1-4), 1-52.

Conrad, D. H., Waldschmidt, T., Lee, W. T., Rao, M., Keegan, A. D., Noelle, R. J., Lynch, R. G., and Kehry, M. R. (1987). Effect of B-cell stimulatory factor-1 (interleukin 4) on Fcε and Fcγ Receptor Expression on murine B Lymphocytes and B cell lines. J Immunol 139, 2290-2296.

Cornell, W. D., Cieplak, P., Bayly, C. I., Gould I. R., Merz Jr. K. M., Frergusson, D. M., Spellmeyer, D. C., Fox, T., Caldwell, J. W., and Kollman, P. A. (1995). A second generation force field for the simulation of proteins and nucleic acids. JACS 117, 5179-5197.

Cregut, D., Serrano, L. (1999). Molecular dynamics as a tool to detect protein foldability. A mutant of domain B1 of protein G with non-native secondary structure propensities. Protein Science 8, 271-282

Crick, F. H. C. (1953). The packing of α-helices: simple coiled-coils. Acta Crystallogr. 6, 689-697.

Cwirla, S. E., Balasubramanian, P., Duffin, D. J., Wagstrom, C. R., Gates, C. M., Singer, S. C., Davis, A. M., Tansik, R. L., Mattheakis, L. C., Boytos, C. M., Schatz, P. J., Baccanari, D. P., Wrighton, N. C., Barrett, R. W. & Dower, W. J. (1997). Peptide agonist of the thrombopoietin receptor as potent as the natural cytokine. Science 276(5319), 1696-9.

Cwirla, S. E., Peters, E. A., Barrett R. W., Dower, D. J. (1990). Peptides on Phage: a vast library of peptides for identifying ligands. Proc. Natl. Acad. Sci USA 87, 6387-6382.

de Vos, A. M., Ultsch, M., Kossiakoff, A. A. (1992). Human Growth Hormone and Extracellular Domain of its Receptor: Crystal structure of the Complex. Science 255, 306-312.

Domingues, H., Cregut, D., Sebald, W., Oschkinat, H. & Serrano, L. (1999). Rational design of a GCN4-derived mimetic of interleukin-4. Nat Struct Biol 6(7), 652-6.

Eastgate, J. A., Symons, J. A. & Duff, G. W. (1990). Identification of an interleukin-1 beta binding protein in human plasma. FEBS Lett 260(2), 213-6.

Finkelman, F. D., Madden, K. B., Morris, S. C., Holmes, J. M., Boiani, N., Katona, I. M., Maliszewski, C. R. (1993). Anti-cytokine antibodies as carrier proteins: prolongation of in vivo effects of exogenous cytokines by injection of cytokine-anti-cytokine antibody complexes. J. Immunol. 151, 1235.

Foa, P. (1994). Erythropoietin: Basic and Clinical aspects. In Acta Haematolol (Spivak, J. L., ed.), Vol. 8, pp. 863. W. B. Saunders, Philadelphia.

Grunewald, S. M., Werthmann, A., Schnarr, B., Klein, C. E., Bröcker, E. B., Mohrs, M., Brombacher, F., Sebald, W., Duschl, A. (1998). An antagonist IL-4 mutant prevents type I allergy in the mouse: inhibition of the IL-4/IL-13 receptor system completely abrogates humoral and immune response to allergen and development of allergic symptoms in vivo. J. Immunol. 160, 4004-4009.

Jacobs, K., Shoemaker, C., Rudersdorf, R., Neill, S. D., Kaufman, R. J., Mufson, A., Seehra, J., Jones, S. S., Hewick, R., Fritsch, E. F. & et al. (1985). Isolation and characterization of genomic and cDNA clones of human erythropoietin. Nature 313(6005), 806-10.

Livnah, O., Stura, E. A., Johnson, D. L., Middleton, S. A., Mulcahy, L. S., Wrighton, N. C., Dover, W. J., Jollife, L. K., Wilson, I. A. (1996). Functional mimicry of a protein hormone by a peptide agonist: The EPO Receptor complex at 2.8 Å. Science 273, 464-471.

Maesaki, R., Ihara, K., Shimizu, T., Kuroda, S., Kaibuchi, K. & Hakoshima; T. (1999). The structural basis of Rho effector recognition revealed by the crystal structure of human RhoA complexed with the effector domain of PKN/PRK1. Mol Cell 4(5), 793-803.

Maliszewski, C. R., Sato, T. A., Davison. B., Jacobs, C. A., Finkelman, F. D., Fanslow, W. C. (1994). In vivo biological effects of soluble interleukin-4 receptor. Proc. Soc. Exp. Biol. Med. 206, 233.

Monera, O. D., Kay, C. M. & Hodges, R. S. (1994). Electrostatic interactions control the parallel and antiparallel orientation of alpha-helical chains in two-stranded alpha-helical coiled-coils. Biochemistry 33(13), 3862-71.

Munson, M., Balasubramanian, S., Fleming, K. G., Nagi, A. D., O'Brien, R., Sturtevant, J. M. & Regan, L. (1996). What makes a protein a protein? Hydrophobic core designs that specify stability and structural properties. Protein Sci 5(8), 1584-93.

O'Shea, E. K., Klemm, J. D., Kim, P. S., Alber, T. (1991). X-ray structure of the GCN4 leucine zipper, a two-stranded, parallel coiled coil. Science 25, 539-544.

Pace, C., N., Vajdos, F., Fee, L., Grimsley, G., Gray, T. (1995). How to measure and predict the molar absorption coefficient of a protein. Protein Science 4, 2411-2423.

Paul, W. E. (1991). Interleukin 4: a prototypic immunoregulatory lymphokine. Blood 77(9), 1859-1870.

Pearlman, D., A., Case, D. A., Caldwell, J. C., Ross, W. S., Cheatham, T., E., Fergusson, D., M., Seibel, G., L., Chancra Singh, U., Weiner, P., Kollman, P. A. (1995). AMBER 4.1. UCSF, University of California, San Francisco, USA.

Peränen, J., Rikkonen, M., Hyvönen, M., Kääriäinen, L. (1996). T7 Vectors with a modified T7lac promoter for expression of proteins in Escherichia coli. Analytical Biociemistry 236, 371-373.

Rose-John, S., Heinrich, P. C. (1994). Soluble receptors for cytokines and growth factors: generation and biological function. Biochem. J. 300, 281-290.

Russel, S. M., Keegan, A. D., Harada, N., Nakamura, Y., Noguchi, M., Leland, P., Friedmann, M. C., Miyajima, A., Puri, R. K., Paul, W. E., Leonard, W. J. (1993). Interleukin-2 Receptor gamma chain: a functional component of the interleukin 4 receptor. Science 262, 1880-1883.

Shen, B.-J., Hage, T.; Sebald, W. (1996). Global and local determinants for the kinetics of interleukin-4/interleukin-4 receptor interaction.

Soisson, S. M., MacDougall-Shackleton, B., Schleif, R. & Wolberger, C. (1997a). Structural basis for ligand-regulated oligomerization of AraC. Science 276(5311), 421-5.

Soisson, S. M., MacDougall-Shacldeton, B., Schleif, R. & Wolberger, C. (1997b). The 1.6 A crystal structure of the AraC sugar-binding and dimerization domain complexed with D-fucose. J Mol Biol 273(1), 226-37.

Tony, H.-P., Shen, B.-J., Reusch, P., Sebald, W. (1994). Design of human interleukin-4 antagonists inhibiting interleukin-4-dependent and interleukin-13-dependent responses in T-cells and B-cells with high efficiency. Eur. J. Biochem. 225, 659-665.

Tuffery, P., Etchebest, C., Hazout, S., Lavery, R. (1991). A new approach to the rapid determination of protein side chain conformation. Journal of Biomolecular structure and dynanics 8(6), 1267-1289.

Twigg, A. J. & Sherratt, D. (1980). Trans-complementable copy-number mutants of plasmid ColE1. Nature 283(5743), 216-8.

van Kimmenade, B., M. W, Schumacher, J. H., Laquoi, C., Kalstelein, R. A. (1988). Expression, renaturation and purification of recombinant interleukin-4 from Escherichia coli. Eur. J. Biochem. 173, 109-114.

Wrighton, N. C., Balasubramanian, P., Barbone, F. P., Kashyap, A. K., Farrell, F., X., Jolliffe, L. K., Barrett, R. W., Dower, W. (1997). Increased potency of an erythropoietn peptide mimetic through covalent dimerization. Nat. Biotech 15, 1261-1265.

Wrighton, N. C., Farrell, F. X., Chang, R., Kashyap, A. K., Barbone, F. P., Mulcahy, L. S., Johnson, D. L., Barrett, R. W., Jolliffe, L. K., Dower, W. J. (1996). Small peptides as potent mimetics of the Protein Hormone Eritropoietin. Science 273, 458-463.

Yanofsky, S. D., Baldwin, D. N., Butler, J. H., Holden, F. R., Jacobs, J. W., Balasubramanian, P., Chinn, J. P., Cwirla, S. E., Peters-Bhatt, E., Whitehorn, E. A., Tate, E. H., Akeson, A., Bowlin, T. L., Dower, W. J. & Barrett, R. W. (1996). High affinity type I interleukin 1 receptor antagonists discovered by screening recombinant peptide libraries. Proc Natl Acad Sci USA 93(14), 7381-6.

Zhou, N. E., Kay, C. M. & Hodges, R. S. (1994). The net energetic contribution of interhelical electrostatic attractions to coiled-coil stability. Protein Eng 7(11), 1365-72.

Zhu, B. Y., Zhou, N. E., Kay, C. M. & Hodges, R. S. (1993). Packing and hydrophobicity effects on protein folding and stability: effects of beta-branched amino acids, valine and isoleucine, on the formation and stability of two-stranded alpha-helical coiled coils/leucine zippers. Protein Sci 2(3), 383-94.

Zurawski, S., Vega, F., Jr., Doyle, E. L., Huyghe, B., Flaherty, K., Mckay, D. B., Zurawski, G. (1993). Definition and spatial location of mouse interleukin-2 residues that interact with its heterotrimeric receptor. EMBO Journal 12(13), 5113-5119.

1. A peptide mimetic of a cytokine molecule comprising an atypical helix-turn-helix motif mutated to incorporate one or more amino acid residues from the active site of said cytokine molecule.

2. A peptide mimetic according to claim 1, wherein said atypical helix-turn-helix motif is derived from the ROP protein (GenBank accession no. P03051); the dimerization domain of the Escherichia coli gene regulatory protein AraC (pdb code 2ara and 2aac); the ACC finger domain of the effector domain of protein kinase PKN/PRK1; or the coiled-coil of Thermus Thermophilus seryl-tRNA synthetase (pdb code 1ser) (Biou et al., 1994).

3. A peptide mimetic according to claim 1 or claim 2, wherein said peptide mimetic comprises a helix-turn-helix motif of the ROP protein.

4. A peptide mimetic according to claim 3, wherein said peptide mimetic comprises a ROP helix-turn-helix monomer.

5. A peptide mimetic according to any one of the preceding claims, wherein residues from the N terminus and/or the C terminus of the atypical helix-turn-helix are deleted.

6. A peptide mimetic according to any one of claims 1-5, wherein the peptide sequence includes a methionine residue at its N terminus.

7. A peptide mimetic according to any one of the preceding claims, wherein said cytokine molecule is a four helix bundle cytokine.

8. A peptide mimetic according to any one of the preceding claims, wherein said four helix bundle cytokine is human growth hormone (HGH), granulocyte Macrophage-Colony stimulating factor (GM-CSF), granulocyte Colony stimulating factor (G-CSF), leukaemia inhibitory factor (LIF), erythropoietin (EPO), IL-2, IL-3, IL-4, IL-5, IL-6, IL-7, IL-9, IL-13, ciliary neurotrophic factor (CNTF), oncostatin (OSM) or an interferon.

9. A peptide mimetic according to claim 8, wherein said four helix bundle cytokine is IL-4, IL-2 or HGH.

10. A peptide mimetic according to claim 9, wherein said peptide mimetic binds to the IL-4 receptor, IL-2 receptor or HGH receptor with an affinity of at least 50 μM.

11. A peptide mimetic according to any one of claims 8-10, wherein said cytokine is IL-4.

12. A peptide mimetic according to claim 11, wherein said atypical helix-turn-helix mutated to incorporate the active site of said cytokine molecule includes one or more of the following substitutions:
Met 11 Ile; Ile 15 Glu; Glu 33 Lys; Ile 37 Arg; Ser 40 Lys; Leu 41 Arg; His 44 Arg; Ala 45 Asn; and Glu 47 Trp.

13. A peptide mimetic comprising the amino acid sequence of solup10 presented in SEQ ID NO:1.

14. A peptide mimetic consisting of the amino acid sequence of solup10 presented in SEQ ID NO:1.

15. A nucleic acid molecule encoding a peptide mimetic according to any one of the preceding claims.

16. A nucleic acid sequence according to claim 15, which comprises or consists of the nucleotide sequence presented as SEQ ID NO: 2 herein.

17. A vector comprising a nucleic acid molecule according to either of claims 15 or 16.

18. A host cell containing a nucleic acid molecule according to either of claims 15 or 16 or a vector according to claim 17.

19. A peptide mimetic according to any one of claims 1-14, for use as a pharmaceutical.

20. The use of a peptide mimetic according to any one of claims 1-14 in the manufacture of a medicament for the treatment or prevention of a disease in a mammal, preferably a human.

21. A method of preventing or treating a disease or condition in a patient, comprising administering a peptide mimetic according to any one of claims 1-14 or a nucleic acid molecule according to claim 15 or claim 16 to the patient in a therapeutically-effective amount.

22. A pharmaceutical composition comprising a peptide mimetic according to any one of claims 1-14, in combination with a pharmaceutically-acceptable carrier.

23. A diagnostic kit comprising a peptide mimetic according to any one of claims 1-14.

24. A transgenic non-human mammal, carrying a transgene encoding a peptide mimetic according to any one of the claims 1-14.

25. A method of generating a peptide mimetic of a cytokine molecule, said method comprising incorporating the binding site of the cytokine molecule into the sequence of an atypical helix-turn-helix motif.

26. A method according to claim 25, additionally comprising a step of mutating the sequence of the generated peptide mimetic and selecting for variants of this sequence with improved biological activity as a mimetic of a cytokine molecule.

27. Use of an atypical helix-turn-helix motif as a template for the design of a peptide mimetic of a cytokine.

28. A method for the preparation of a cytokine receptor comprising passing a composition containing the cytokine receptor over a matrix to which a peptide mimetic according to the invention is bound.

29. A method according to claim 28, wherein said cytokine receptor is IL-4Rα.

30. A method for the preparation of an antibody against a cytokine comprising immunising an animal with a peptide mimetic according to any one of claims 1-14.
